(19) United States
(12) Patent Application Publication
(10) Pub. No.: US 2026/0035451 A1
(43) Pub. Date: Feb. 5, 2026

(54) PROCESS FOR MANUFACTURING ANTIBODY FRAGMENT PROTEIN

(71) Applicant: Lupin Limited, Mumbai (IN)

(72) Inventors: Ashok Kumar Mishra, Pune (IN); Ankit Srivastava, Pune (IN); Ajinath Navnath Tambe; Mohammad Shahnawaz, Pune (IN); Shivani Jain, Pune (IN); Sanjay Kumar Tiwari, Pune (IN)

(21) Appl. No.: 19/167,160

(22) PCT Filed: Mar. 1, 2024
(86) PCT No.: PCT/IB2024/051993

§ 371 Date: Sep. 19, 2025

(30) Foreign Application Priority Data
Mar. 1, 2023 (IN) 202221069433

(51) Int. Cl. C07K 16/24

(52) U.S. Cl. CPC C07K 16/241; C07K 2317/40; C07K 2317/55

(57) ABSTRACT

The invention provides a process for manufacturing certolizumab pegol from bacterial host cells. The invention provides refolding process of certolizumab wherein the solubilized solution of heavy chain and light chain of certolizumab is treated with refolding buffer under suitable conditions including pII, temperature, and incubation period to obtain high quality and quantity of refolded protein. The invention further provides efficient pegylation process to obtain certolizumab pegol in good yields.

### FIELD OF THE INVENTION

The present invention relates to the process for the manufacturing of certolizumab pegol. More specifically, the invention relates to processes for the rcfolding of recombinantly produced certolizumab from inclusion bodies formed in bacterial cells. Further, the invention also relates to site-specific pegylation process to obtain certolizumab pegol.

### BACKGROUND OF THE INVENTION

Antibody fragments are smaller than conventional antibodies and generally lack glycosylation, which allows their production in prokaryotic expression systems. Currently, there are several antibody fragments approved for therapeutic purposes. Some of the approved antibody fragments are certolizumab, blinatumomab, capacizumab, ranibizumab, brolucizumab, etc. Further, some of the approved antibody fragments are conjugated with effector molecules such as branched polyethylene glycol compounds.

Certolizumab pegol, a TNF blocker is a recombinant, humanized antibody Fab′ fragment, with specificity for human tumor necrosis factor alpha (TNFα), conjugated to an approximately 40 kDa polyethylene glycol (PEG2MAL40K). The Fab′ fragment is manufactured in E. coli and is subsequently subjected to conjugation to PEG2MAL40K, to generate certolizumab pegol. The Fab′ fragment is composed of a light chain with 214 amino acids and a heavy chain with 229 amino acids. The molecular weight of certolizumab pegol is approximately 91 kiloDaltons.

Although gene constructs of the smaller antibody fragments such as certolizumab are readily made, expression of folded fragments turns out to be rather difficult duc to the non-native structures, where they can be readily made in bacterial cells as inclusion bodies (IBs), provided it can be successfully refolded. However, the formation of inclusion bodies and refolding of the insoluble proteins have been major obstacles in fully utilizing Escherichia coli cytoplasmic expression technologies. Inclusion bodies contain mainly inactive misfolded protein in an insoluble form and converting it into an active native conformation has always been a challenge.

U.S. Pat. No. 8,378,073 discloses the process to attach effector molecules such as 40 kDa polyethylene glycol to a protein via a reactive group in the protein such as cysteine.

One of the important challenges in the development of such antibody fragment proteins is to identify efficient and competent refolding processes for large-scale production. Refolding of proteins is typically a multistep process, wherein protein denaturation and renaturation to gain properly folded protein is the key step to maximize the efficiency of refolding process. Criticality of the denaturation and renaturation step increases with the complexity of molecules like Fab fragments such as certolizumab, which requires a longer refolding time to get the optimal refolding yield. There is a need to develop an efficient process for the manufacturing of certolizumab pegol.

### SUMMARY OF THE INVENTION

In an aspect the invention provides a process for producing certolizumab pegol, the process comprising the steps of:
a) isolating inclusions bodies comprising light chain and heavy chain of certolizumab expressed in bacterial host cells;
b) refolding the light chain and heavy chains of certolizumab to obtain certolizumab;
c) optionally purifying the certolizumab obtained from step b);
d) PEGylating certolizumab with 40 kD PEG to obtain certolizumab pegol; and
e) optionally purifying the certolizumab pegol obtained from step d).

In another aspect the invention provides a process for producing certolizumab, the process comprising the steps of:
a) isolating inclusions bodies comprising light chain and heavy chain of certolizumab expressed in bacterial host cells;
b) refolding the light chain and heavy chains of certolizumab to obtain certolizumab; and
c) optionally purifying the certolizumab obtained from step b).

In yet another aspect the invention provides a process for producing certolizumab pegol, the process comprising the steps of:
a) treating certolizumab with reducing agent and chelating agent;
b) incubating the solution obtained from step a) for 30 to 180 min;
c) concentrating the solution and adding 40 kD methoxy PEG maleimide at pH 4.5±1 to obtain certolizumab pegol; and
d) optionally purifying the certolizumab pegol obtained from step c).

In yet another aspect the invention provides a process for refolding certolizumab, the process comprising the steps of:
a) isolating inclusions bodies comprising light chain and heavy chain of certolizumab expressed in bacterial host cells;
b) solubilizing the inclusion bodies in solubilization buffer and reducing agent;
c) incubating for 20 to 120 min;
d) diluting the solubilized solution with a refolding buffer slowly over 45±30 min;
e) immediately adjusting the pH of the solution to 8 to 10;
f) incubating the solution obtained from step e) for 6 to 16 hrs; and
g) concentrating the solution to obtain certolizumab; and
h) optionally purifying the certolizumab obtained.

In another aspect the invention provides a process for producing pegylated protein, the process comprising the steps of:
a) purifying protein expressed in bacterial host cells by first chromatography;
b) pegylating protein obtained from step a); and
c) purifying pegylated certolizumab by second chromatography;
   wherein the buffer used in step a), b) and c) have same anionic and cationic components.

In one aspect the invention provides for producing pegylated protein, the process comprising the steps of:
d) purifying protein expressed in bacterial host cells by first chromatography;
e) pegylating protein obtained from step a); and
f) purifying pegylated certolizumab by second chromatography;
   wherein the buffer used in step a), b) and c) have same anionic and cationic components.

In yet another aspect the invention provides a process for producing certolizumab pegol, the process comprising the steps of:
a) purifying certolizumab expressed in bacterial host cells by first ion exchange chromatography;
b) pegylating certolizumab obtained from step a); and
c) purifying pegylated certolizumab by second ion exchange chromatography;
   wherein the buffer used in step a), b) and c) have same anionic and cationic components.

### DESCRIPTION OF THE DRAWINGS

FIG. 1: RP-HPLC profile of solubilized and reduced inclusion bodies obtained according to example 3.

FIG. 2: RP-HPLC profile of refolded Fab′ fragment of certolizumab obtained according to example 3.

FIG. 3: SE-HPLC profile of Pegylated Fab′ fragment obtained according to example 6.

FIG. 4: SE-HPLC profile of Pegylated Fab′ fragment obtained according to example 7.

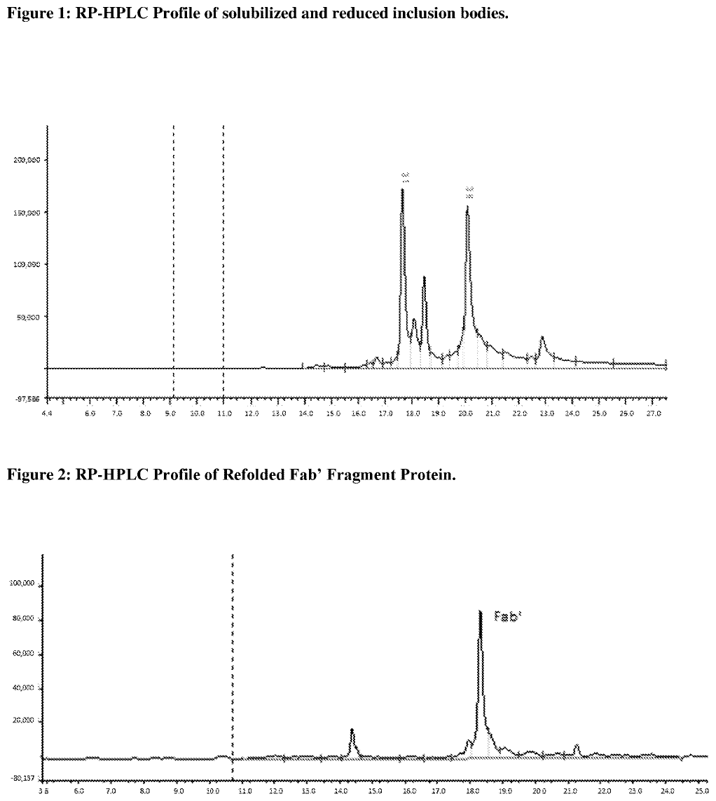

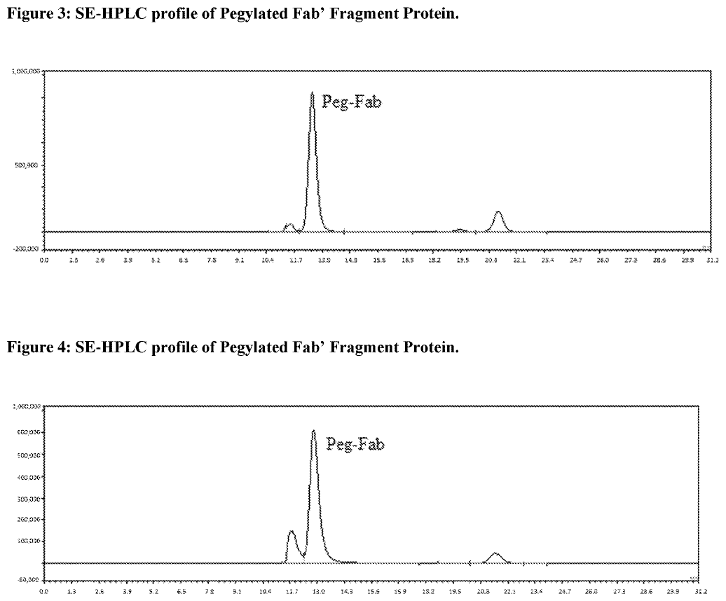

### DETAILED DESCRIPTION OF THE INVENTION

### Definitions

“Anion exchange resin” or “Anion exchange chromatography” mentioned in the embodiments refers to a solid chromatographic support, which has a positively charged ligand such as a quaternary amino group attached thereto, capable of ionic interaction with a negatively charged protein or a functional group under suitable conditions. The anion exchange resin can be any weak or strong anion exchange resin or a membrane, which could function as a weak or a strong anion exchanger. Various commercially available anion exchange resins are known in the art and include without any limitation DEAE cellulose, Poros PI 20, PI 50, HQ 10, HQ 20, HQ 50, D 50 from Applied Biosystems, MonoQ, MiniQ, Source 15Q and 30Q, Q, DEAE and ANX Sepharose Fast Flow, Q Sepharose high Performance, QAE SEPHADEX and FAST Q SEPHAROSE from GE Healthcare, Macro-Prep DEAE and Macro-Prep High Q from Biorad, Q-Ceramic Hyper D. DEAE-Ceramic Hyper D. from Pall Corporation.

As used herein, “antibody's fragment” or “Fab Fragment protein” refers generally to the antigen binding fragments of antibodies selected from the group comprising Fab fragment, F(ab′)₂ fragment and Fab′ fragment. Antibody fragments are expressed as heterologous protein in to the host cell are having more than about ten amino acids. Examples of antibody's fragments are including but not limited to certolizumab, blinatumomab, caplacizumab, ranibizumab, brolucizumab, abciximab, anatumomab, arcitumomab, bectumomab, biciromab, naptumomab, nofetumomab, sulesomab, tadocizumab, telimomab, etc.

As used herein, the term “bind-elute mode” refers to a mode of purification by chromatography, wherein the Protein-of-interest when loaded on the column is bound to the chromatographic resin and is subsequently eluted with an elution buffer.

As used herein, “buffer” or “buffered solution” refers to a solution which resists changes in pH by the action of its acid-base conjugate components.

As used herein, “chaotropic agent” refers to a compound that, in a suitable concentration in aqueous solution, is capable of changing the spatial configuration or conformation of polypeptides through alterations at the surface thereof so as to render the polypeptide soluble in the aqueous medium. The alterations may occur by changing, e.g. the state of hydration, the solvent environment, or the solvent-surface interaction. The concentration of chaotropic agent will directly affect its strength and effectiveness. A strongly denaturing chaotropic solution contains a chaotropic agent in large concentrations which, in solution, will effectively unfold a polypeptide present in the solution effectively eliminating the proteins secondary structure. The unfolding will be relatively extensive, but reversible. A moderately denaturing chaotropic solution contains a chaotropic agent which, in sufficient concentrations in solution, permits partial folding of a polypeptide from whatever contorted conformation the polypeptide has assumed through intermediates soluble in the solution, into the spatial conformation in which it finds itself when operating in its active form under endogenous or homologous physiological conditions. Examples of chaotropic agents include one or more of guanidine hydrochloride, urea, and hydroxides such as sodium or potassium hydroxide and mixtures thereof.

The term “dual cistronic expression system” as used herein includes a polynucleotide sequence encoding a polypeptide to be expressed and sequences controlling its expression such as a promoter and optionally an enhancer sequence.

The promoter of the invention is either operably linked to the gene to be expressed, i.e. transcription unit, or is separated therefrom by intervening DNA such as for example by the 5′-untranslated region of the heterologous gene. Preferably the expression system is flanked by one or more suitable restriction sites to enable the insertion of the expression cassette into a vector and/or its excision from a vector. The promoter may be selected from T7 promoter, arabinose promoter phoA, tac, lpp, lac-lpp, lac, trp, trc, preferably T7 promoter and arabinose promoter. In certain embodiment the dual cistronic expression system comprises two cistrons expressing polynucleotide sequence encoding protein of interest under the control of two promoters.

In one embodiment the dual cistronic expression system comprises:
a) first cistron comprises T7 promoter operably linked with polynucleotide sequence encoding heavy chain of certolizumab;
b) second cistron comprises arabinose promoter operably linked with polynucleotide sequence encoding light chain of certolizumab;
wherein the first and second cistrons are positioned in single vector and express the heavy chain and light chain of the certolizumab in bacterial cell.

In some embodiment the position of first and second cistron is interchangeable wherein the second cistron may be cloned in vector at the position of first cistron and first cistron may be positioned at second cistron. The heavy chain and light chain of certolizumab independently express as inclusion bodies and may be further treated to obtain certolizumab.

The term “gradient elution” is used herein to refer generally to conditions in which pH and/or conductivity is either increased or decreased using at least two buffers wherein the buffers are different in terms of pH or conductivity or both.

The term “linear gradient” is used here to refer to conditions in which pH and/or conductivity is either increased or decreased gradually using at least two buffers wherein the buffers are different in terms of pH or conductivity or both.

As used herein, the term “misfolded” protein refers to precipitated or aggregated polypeptides that are contained within inclusion bodies. As used herein, “insoluble” or “misfolded” Fab fragment protein such as certolizumab or other recombinant protein refers to precipitated or aggregated protein that is contained within the periplasm or intracellular space of prokaryotic host cells and assumes a biologically inactive conformation with mismatched or unformed disulfide bonds. The insoluble recombinant protein is generally, but need not be. contained in inclusion bodies.

The terms “purifying” or “purified” or “separating,” or “isolating,” or purification” or “separation” as used interchangeably herein, refer to increasing the degree of purity of a polypeptide or protein of interest or a target protein from a protein mixture comprising the polypeptide and one or more impurities or contaminants including at least one of the product related impurities.

As used herein, “reducing agent or reductant” refers to a compound that, in a suitable concentration in aqueous solution, maintains free sulfhydryl groups so that the intra-or intermolecular disulfide bonds are chemically disrupted. Representative examples of suitable reducing agents include dithiothreitol (DTT), dithioerythritol (DTE), beta-mercaptoethanol (BME), cysteine, cysteamine, thioglycolate, glutathione, and sodium borohydride.

As used herein, “solubilization buffer” refers to a solution used for solubilization of inclusion bodies.

In certain embodiments, the processes and procedures are applicable to manufacturing or industrial scale production, refolding, and purification of the recombinant certolizumab protein.

In certain embodiments, the processes and procedures are applicable to manufacturing or industrial scale production, refolding, pegylation and purification of the recombinant certolizumab pegol.

The present invention provides a process to recover the refolded certolizumab protein from insoluble proteins formed in intracellular or in the periplasmic space bacterial host cell. The inclusion bodies are isolated from the prokaryotic cell as per the process known in the art. The isolated inclusion bodies mainly comprise misfolded insoluble proteins and impurities.

In an embodiment the protein expressed in bacterial cells as inclusion bodies or the cell lysate are solubilized in a buffered solution comprising chaotropic agent and/or reducing agent. The solubilization buffer is selected from the group comprising of but not limited to Tris-base buffers, or the buffer comprises Tris-Cl buffer having pH from about 8 to 12.

In another embodiment the chaotropic agent is selected from the group comprising but not limited to guanidine hydrochloride, urea, sodium dodecyl sulphate or deoxycholic acid. In one embodiment the concentration of urea is between about 3 M to about 10 M. In another embodiment the reducing agent for the refolding process is dithiothreitol (DTT).

In one embodiment of the invention, the light chain and heavy chains of the certolizumab are solubilized with Tris buffer, urea and DTT.

In yet another embodiment the solubilized protein solution is stabilized with a stabilizer in water. By way of dilution with addition of the stabilizer solution in water, the concentration of chaotropic agents such as urea is lowered, thereby it stabilizes the solubilized protein. The stabilizer is selected from the group comprising of but not limited to arginine, lysine, or glycine. In one more embodiment of the invention, the said process does not involve the step of pH adjustment or regulating the pH during dilution.

In one embodiment of the invention, the solubilized protein solution is diluted with one or more stabilizers in water to 10-30 fold dilution so that the concentration of chaotropic agent such as urea is reduced in the range of less than 0.3M to 1M Subsequently, redox components such as cystine and cysteine is added to the solubilized protein solution. In one embodiment 2-4 mM of cystine, and about 0.4-0.8 mM cysteine, is added to the solution. In one embodiment 1-10 mM of cystine, and about 0.2-1.0 mM cysteine, is added to the solution.

In one more embodiment of the invention, refolding of the protein is triggered by adjusting the pH of the obtained diluted refolding mixture. In one embodiment of the invention, pH of the solubilized protein is adjusted to 8-10. The pH of the solution is adjusted with HCl or arginine HCl.

In one more embodiment of the invention, the efficacy of refolding process according to the present invention is in the range of 25% to 85%. The refolding of the protein according to the present invention is achieved within a time span of 6 to 12 hours.

In one more embodiment the invention provides a process for recovering refolded certolizumab, the process comprising the steps of:
a. isolating inclusion bodies comprising light chain and heavy chain of the certolizumab from the bacterial host cell;
b. solubilizing the light chain and heavy chains of certolizumab in a buffered solution comprising chaotropic agent and reducing agent to obtain solubilized protein solution;
c. diluting the solubilized solution with one or more stabilizer such as arginine, lysine or glycine base in water to obtain refolding mixture;
d. refolding the light chain and heavy chain of the certolizumab by reducing the pH of the refolding mixture; and
e. recovering the refolded certolizumab.

In one more embodiment, the invention provides a process for purification of the refolded certolizumab protein. The refolded protein obtained according to the present invention is further subjected to the chromatographic processes including but not limited to affinity chromatography step such as protein-A, protein-L or protein-G chromatography and/or one or more combinations of chromatography steps such as anion or cation exchange chromatography, hydrophobic interaction chromatography and mixed mode chromatography. The obtained refolded certolizumab protein is further optionally subjected to one or more steps of buffer exchange and/or ultrafiltration/tangential flow filtrations to obtain the purified protein. The protein of interest may be obtained in bind-elute or flow through mode during the chromatography purification step.

In one more embodiment of the invention, the Fab protein obtained according to present invention is conjugated with branched polymers such as branched polyethylene glycol compounds. The concentrated fab protein is diluted with a buffer containing reductant in a batch mode. The reductant used for activation is selected from the group comprising of but not limited to mild thiol based reductants such as β-mercaptoethanol, glutathione, cysteine, cysteamine (β-mercaptoethylamine). Subsequently, the reductant is removed by diafiltration with a suitable buffer followed by concentration of reduced fab protein to facilitate pegylation. The pegylation of Fab protein is carried out by addition of equimolar amount of activated PEG compound such as 40 KDa Methoxy PEG maleimide to the certolizumab solution.

In one more embodiment of the invention, the certolizumab protein obtained according to present invention is conjugated with branched polymers such as branched polyethylene glycol compounds. Reductant is added in the column eluate and incubated for 30 minutes to 2 hours after adjusting the pH of the eluate to pH 6.8±0.4. Alternatively the reductant is added in the column eluate and incubated for 30 minutes to 2 hours after adjusting the pH of the eluate to pH 5 to 8. Subsequently, the reductant is removed by diafiltration with a suitable buffer followed by concentration of reduced protein to facilitate pegylation. The pegylation of certolizumab protein is carried out by addition of equimolar amount of activated PEG compound such as 40 KDa methoxy PEG maleimide to the certolizumab protein solution.

In one embodiment of the invention, the pegylation process according to present invention results in 50 to 80% site specific attachment of branched monomethyl PEG such as 40 KDa methoxy PEG maleimide, 8 to 12 hours of incubation.

In another embodiment of the invention, the pegylation process according to present invention results in more than 50% site specific attachment of branched monomethyl PEG such as 40 KDa methoxy PEG maleimide, with 6 to 16 hours of incubation.

In one more embodiment of the invention, the pegylated certolizumab obtained according to present invention is further subjected to one or more chromatography steps and/or buffer exchange, ultrafiltration/tangential flow filtrations to obtain the purified form of pegylated protein. The chromatography columns may be selected from anion or cation exchange, hydrophobic interaction or mixed mode chromatography columns.

The pegylated certolizumab may be obtained in bind-elute or flow through mode during the chromatography purification step.

In one embodiment, the invention provides a process for recovering refolded Fab protein of interest, the process comprising the steps of:
a. isolating a light chain and heavy chain of the protein of interest expressed in bacterial host cells;
b. solubilizing the light chain and heavy chains of the protein of interest in a buffered solution comprising chaotropic agent and reducing agent to obtain solubilized protein solution;
c. diluting the solubilized solution with one or more stabilizer such as arginine, lysine or glycine in water to obtain refolding mixture;
d. refolding the light chain and heavy chains of the protein of interest by reducing the pH of the refolding mixture; and
e. recovering the refolded protein of interest.

The examples which follow are illustrative of the invention and are not intended to be limiting.

Those having ordinary skill in the art will clearly understand that many modifications are possible in the embodiments and examples without departing from the teachings thereof.

### Example-1: Expression of Protein

The E. coli strain BL21-A1 cell line was used as the expression host. The light chain and heavy chain of certolizumab as described EP1287140 were cloned in the dual cistron vector containing two expression cassettes, one each for light chain and heavy chain. The details of dual cistron vector are available elsewhere in WO2016005931. The heavy and light chain genes were expressed under T7 and arabinose promoters separately. The expressed protein was in the form of inclusion bodies, in the periplasmic space of E. coli.

### Example 2: Refolding of Certolizumab Protein

Post harvest, cells are lysed in presence lysis buffer (tris, EDTA and urea at pH 8.0). Once cell lysis is completed, pH of the solution is adjusted to 11.0 in and reducing agent is added (DDT) and solution is incubated at room temperature for 30 minutes under stirring condition for reducing the protein.

Protein is then diluted (10-15 folds) with refolding buffer (0.6M L-arginine) and pH is adjusted to pH 9.0 using HCl. This solution is incubated for 6-8 hours at room temperature under stirring condition.

Post incubation, protein solution is filtered through hollow fiber membrane followed by buffer exchange.

### Example 3: Refolding of Certolizumab Protein

The protein is expressed in Example-1 is an insoluble form, which is then isolated by continuous centrifugation using disk stack centrifuge. The obtained crude inclusion bodies are then solubilized with 50 mM Tris-Cl, 8M urea and 4 mM DTT. The obtained solution is diluted 25×, without adjusting the pH with 0.6M Arginine-base in water, 3 mM Cystine and 0.6 mM Cysteine over period of 45±15 minutes. Immediately after the addition of cysteine/cysteine and Arginine in water, the pH of the refolding solution was adjusted to 9.0 with 6N HCl. The said refolding solution is then incubated at room temperature for about 8 hrs under stirring condition. The profile of refolded protein is then analyzed through RP-HPLC. Yield: 44%.

### Example 4: Purification of Refolded Certolizumab

Refolded certolizumab obtained from Example 2 or 3 was concentrated and purified using Mixed mode chromatography (Capto MMC). The concentrated protein was loaded to column using loading buffer of 50 mM Sodium acetate pH 4.7±0.2. Column was washed using 2-4 CV of loading buffer. The certolizumab was eluted using a step gradient of elution buffer (25 mM Sodium Phosphate pH 6.7±0.2).

### Example 5: Purification of Refolded Certolizumab

The pH of eluted protein from Example 4 adjusted to pH 4.7±0.2 and was purified using Cation exchange chromatography. The protein was loaded to the cation exchange column (Fractogel SO3—) using loading buffer of 25 mM Sodium acetate pH 4.7±0.2. After loading was completed 4-5 CV of wash-buffer (25 mM Sodium acetate+75 mM NaCl pH 4.7±0.2) was used to wash the column. The elution was performed by passing linear gradient of 25 mM Sodium acetate+75 mM NaCl pH 4.7±0.2 and 25 mM Sodium acetate+250 mM NaCl pH 4.7.

### Example 6: Pegylation of Certolizumab Protein

The refolded certolizumab was subjected to cation exchange chromatography and the obtained eluate was concentrated to 25 mg/ml. Protein sample was then diluted with reductant buffer (100 mM sodium phosphate buffer, 5 mM cysteamine hydrochloride, 2 mM EDTA pH 6.8) to 10 folds (v/v) under stirring condition. The dilution of the protein was performed in 10-40 minutes. Once diluted, protein was incubated for 30-180 minutes at room temperature under stirring condition. Then the protein was concentrated up to 10 folds and then buffer exchanged with 20 mM sodium acetate pH 4.5. After the buffer exchange, 40 kDa Methoxy PEG maleimide was added to the protein solution in the molar ratio of 1:1. Obtained pegylation reaction mixture was incubated for 8 hours under stirring condition at room temperature. The profile of pegylated certolizumab is analyzed with SE-HPLC. Yield: 77.81%

### Example 7: Pegylation of Certolizumab Protein

To the eluate of certolizumab from example-5, reducing agent and chelating agent was added to the final concentration of 2.5 mM and 2 mM respectively. pH of the protein mixture was adjusted to 6.8 and then incubated for 30-180 minutes at room temperature and under stirring condition. The protein solution was concentrated up to 20 folds and then buffer exchanged with 20 mM sodium acetate pH 4.5 after incubation. Post buffer exchange, 40 kDa methoxy PEG maleimide was added to the protein solution in the molar ratio of 1:1. The obtained reaction mixture was incubated for 8 hours under stirring condition at room temperature. Pegylation efficiency was evaluated by SE-HPLC. Yield: 75.03%.

### Example 8: Purification of Pegylated Certolizumab

Pegylated certolizumab was purified using cation exchange chromatography by loading the pegylated protein to cation exchange column (Macrocap SP) at pH of 3.8±0.10. Column was washed with 2-6 CV of equilibration buffer (75 mM sodium acetate pH 3.8±0.2.). The pegylated certolizumab was eluted using gradient of 75 mM sodium Acetate+250 mM NaCl pH 3.8±0.2.

1. A process for producing certolizumab pegol, the process comprising steps of:
a) isolating inclusions bodies comprising light chain and heavy chain of certolizumab expressed in bacterial host cells;
b) refolding the light chain and heavy chains of certolizumab to obtain certolizumab;
c) optionally purifying the certolizumab obtained from step b);
d) pegylating certolizumab with activated 40 kD PEG compound to obtain certolizumab pegol; and
e) optionally purifying the certolizumab pegol obtained from step d).

2. A process for producing certolizumab, the process comprising steps of:
a) isolating inclusions bodies comprising light chain and heavy chain of certolizumab expressed in bacterial host cells;
b) refolding the light chain and heavy chains of certolizumab to obtain certolizumab; and
c) optionally purifying the certolizumab obtained from step b).

3. A process for producing certolizumab pegol, the process comprising steps of:
a) treating certolizumab with reducing agent and chelating agent;
b) incubating the solution obtained from step a) for 30 to 180 min;
c) concentrating the solution and adding 40 kD methoxy PEG maleimide at pH 4.5±1 to obtain certolizumab pegol; and
d) optionally purifying the certolizumab pegol obtained from step c).

4. The process as claimed in claim 1, wherein the refolding process of certolizumab comprises the steps of:
a) isolating inclusions bodies comprising light chain and heavy chain of certolizumab expressed in bacterial host cells;
b) solubilizing the inclusion bodies in solubilization buffer and reducing agent;
c) incubating for 20 to 120 min;
d) diluting the solubilized solution with a refolding buffer slowly over 45±30 min;
e) immediately adjusting the pH of the solution to 8 to 10;
f) incubating the solution obtained from step e) for 6 to 16 hrs;
g) concentrating the solution to obtain certolizumab; and
h) optionally purifying the certolizumab obtained from step g).

5. The process as claimed in claim 1, wherein the certolizumab refolded in step b) is purified in step c) by using one or more column chromatographies.

6. The process as claimed in claim 1, wherein the pegylated certolizumab is purified by using one or more column chromatographies.

7. The process as claimed in claim 6, wherein the column chromatographies are selected from ion exchange chromatography, mixed mode chromatography or hydrophobic chromatography.

8. The process as claimed in claim 7, wherein the column chromatography is cation exchange chromatography or mixed mode chromatography.

9. The process as claimed in claim 2, wherein the purified certolizumab is obtained in flow through or bind-elute mode.

10. The process as claimed in claim 3, wherein the reducing agent is cysteamine.

11. The process as claimed in claim 1, wherein the activated 40 kD PEG compound is 40 kD methoxy PEG maleimide.

12. A process for producing pegylated protein, the process comprising the steps of:
a) purifying protein expressed in bacterial host cells by first chromatography;
b) pegylating protein obtained from step a); and
c) purifying pegylated certolizumab by second chromatography;
wherein the buffer used in step a), b) and c) have same anionic and cationic components.

13. The process as claimed in claim 12, wherein the pegylated protein is certolizumab pegol.

14. The process as claimed in claim 12, wherein the first and/or second chromatography is ion exchange chromatography.

15. The process as claimed in claim 12, wherein the first and/or second chromatography is cation exchange chromatography.

16. The process as claimed in claim 2, wherein the refolding process of certolizumab comprises the steps of:
a) isolating inclusions bodies comprising light chain and heavy chain of certolizumab expressed in bacterial host cells;
b) solubilizing the inclusion bodies in solubilization buffer and reducing agent;
c) incubating for 20 to 120 min;
d) diluting the solubilized solution with a refolding buffer slowly over 45±30 min;
e) immediately adjusting the pH of the solution to 8 to 10;
f) incubating the solution obtained from step e) for 6 to 16 hrs;
g) concentrating the solution to obtain certolizumab; and
h) optionally purifying the certolizumab obtained from step g).

17. The process as claimed in claim 2, wherein the certolizumab refolded in step b) is purified in step c) by using one or more column chromatographies.

18. The process as claimed in claim 3, wherein the pegylated certolizumab is purified by using one or more column chromatographies.

19. The process as claimed in claim 18, wherein the column chromatographies are selected from ion exchange chromatography, mixed mode chromatography or hydrophobic chromatography.
